(19) United States
(12) Patent Application Publication
(10) Pub. No.: US 2023/0270712 A1
(43) Pub. Date: Aug. 31, 2023

(54) NOBILETIN COMPOSITIONS AND METHODS FOR THE TREATMENT AND PREVENTION OF COVID-19 AND RELATED PATHOLOGIES

(71) Applicant: CAS PROPERTIES, LLC, Newport Beach, CA (US)

(72) Inventors: James ROUFS, Newport Beach, CA (US); Paul HWANG, Newport Beach, CA (US); Feng ZHAO, Newport Beach, CA (US)

(21) Appl. No.: 18/006,902

(22) PCT Filed: Jul. 26, 2021
(86) PCT No.: PCT/US2021/043226

§ 371 Date: Jan. 26, 2023

Related U.S. Application Data
(60) Provisional application No. 63/057,273, filed on Jul. 27, 2020.

(51) Int. Cl. A61K 31/352; A61K 31/202

(52) U.S. Cl. CPC A61K 31/352; A61K 31/202

(57) ABSTRACT

Compositions and methods for the treatment of COVID-19 in a subject infected with severe acute respiratory syndrome coronavirus 2 (SARS-CoV-2). The methods may include, among other embodiments, administering to the subject a therapeutically effective amount of a composition or formulation comprising nobiletin or salt, solvate, or prodrug thereof.

### CROSS-REFERENCE TO RELATED APPLICATIONS

This application claims the benefit of priority to U.S. Provisional Application Ser. No. 63/057,273, entitled “Nobiletin Compositions and Methods for the Treatment and Prevention of COVID-19 and Related Pathologies,” filed Jul. 27, 2020, the contents of which is hereby incorporated by reference, for all purposes, in its entirety.

### FIELD

The present disclosure is broadly concerned with compositions and methods for the prevention and treatment of viral respiratory infections, including the cold, flu, coronaviruses, and more specifically, the disease, COVID-19, caused by the novel coronavirus known as severe acute respiratory syndrome coronavirus 2 (SARS-CoV-2). The disclosure is also concerned with compositions and methods for the treatment of subjects having and/or diagnosed with pneumonia, acute respiratory failure, or a respiratory infection caused by SARS-CoV-2; a symptomology known as COVID-19.

### BACKGROUND

A new strain of coronaviruses (CoVs) named SARS-CoV-2 (COVID-19 virus) was identified in December 2019 and resulted in a massive outbreak initially in Wuhan, China and propagated to different nations around the globe. In March 2020 the resulting disease, named COVID-19, was declared a pandemic by the World Health Organization (WHO). As of Jul. 3, 2020, there were 10,906,822 confirmed cases of COVID-19 worldwide along with 522,112 recorded deaths for a current mortality rate of 4.79%. In the United States alone, there were 2,741,869 recorded cases and 128,783 recorded deaths for a mortality rate of 4.70%.

With a dearth of positive findings from currently available drugs and results showing that all current drugs are far from a cure, there exists an enormous void in this area of medicine and nutrition. Current evidence also shows that the various vaccines under development may not provide a long-term cure to COVID-19. The United States Centers for Disease Control (CDC) has articulated the potential deficiencies of a COVID-19 vaccine, noting that even when the annual flu vaccine is specific for the circulating influenza virus, the vaccine is far from a cure but instead only reduces the risk of influenza 40%-60%, at best. Therefore, there remains a need for additional therapeutic treatment compositions and methods for the treatment and prevention of COVID-19 and its related pathologies, such as pneumonia.

According to the National Center for Immunization and Respiratory Diseases (NCIRD), high-risk categories for severe illness from COVID-19 includes individuals aged 65 years and older, immunocompromised individuals, those who live in a nursing home or long-term care facility, and people with underlying medical conditions, particularly if not well-controlled. Relevant underlying medical conditions include chronic lung diseases, serious heart conditions, especially hypertension, type 1 and type 2 diabetes, liver disease, chronic kidney disease undergoing dialysis, and severe obesity (BMI of 40+). COVID-19 presents with symptoms that can be far more severe than the typical flu, with pneumonia being one of the greatest contributors to mortality from COVID-19.

According to the CDC, individuals with COVID-19 pneumonia experience the following changes in their lungs: (1) inflammation that may be so severe it damages the lungs' alveoli; (2) fluid accumulation in the lungs; (3) gas exchange difficulties that make it difficult to get enough oxygen or expel enough carbon dioxide; (4) fluid leaking out of blood vessels in the lungs. One of the most important physiological mechanisms underlying the severity of lung disease in COVID-19 is the “cytokine storm” which can lead to acute respiratory distress syndrome or even multiple organ failure.

### DETAILED DESCRIPTION

The present disclosure provides compositions and methods for the treatment and prevention of COVID-19 in a subject as well as the treatment of subjects having an infection caused by severe acute respiratory syndrome coronavirus 2 (SARS-CoV-2). As used herein, the term “severe acute respiratory syndrome coronavirus 2 (SARS-CoV-2),” in all its forms, refers to any natural or laboratory-created variant of the SARS-CoV-2 virus. The present disclosure is also concerned with compositions and methods for the treatment of subjects having and/or diagnosed with pneumonia, acute respiratory failure, or a respiratory infection caused by SARS-CoV-2, as well as the prevention of pneumonia and related pathologies in COVID-19 patients.

It has been surprisingly discovered that compositions comprising a specific phytonutrient, nobiletin, are effective in the prevention of COVID-19 infection in a subject and/or reducing the severity of symptoms of COVID-19 and related pathologies, in particular, inflammation and acute lung injury. As used herein, the term “COVID-19 infection,” in all its forms, refers to an infection caused by severe acute respiratory syndrome coronavirus 2 (SARS-CoV-2), including any variants of the SARS-CoV-2 virus that may naturally arise or that may result from modification of the SARS-CoV-2 in a lab. Nobiletin (5,6,7,8,3′,4′-hexamethoxyflavone) is a polymethoxylated flavone (PMF) found in citrus peels and characterized by the following structural formula:

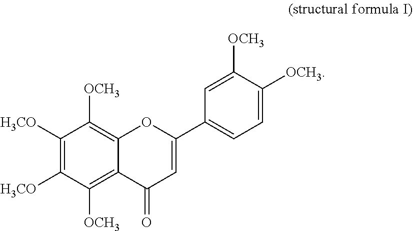 [SMILES: COc1ccc(-c2cc(=O)c3c(OC)c(OC)c(OC)c(OC)c3o2)cc1OC]

Unlike many phytonutrients, it has been discovered that nobiletin has high relative bioavailability, high tissue penetration, and potent anti-viral and anti-inflammatory effects. Additionally, nobiletin has shown excellent ability to restore lung function in subjects having severe pneumonia, including those having severe pneumonia resulting from COVID-19.

Without being held to any particular theory, nobiletin and compositions comprising nobiletin may be effective for the treatment and prevention of viral respiratory infections, including the cold, flu, and coronaviruses, as well as the treatment and prevention of COVID-19 and related pathologies as a result of one or more of the following characteristics and effects of nobiletin: (1) direct anti-viral effects; (2) immunomodulatory effects; (3) anti-inflammatory effects; (4) anti-obesity effects; (5) anti-platelet effects; (6) lung-preserving effects; and (7) tissue penetration characteristics. Nobiletin has been shown to exhibit immunomodulatory effects including potentiating the anti-viral activity of natural killer (NK) cells, key innate immune cells which function as the first line of defense against viruses. One of the mechanisms by which nobiletin can increase the anti-viral activity of NK cells is by increasing 5′ adenosine monophosphate-activated protein kinase (AMPK) levels and activity. Therefore, nobiletin may help prevent COVID-19 infection when administered as a prophylactic composition.

Nobiletin also exhibits direct anti-viral activity. For example, nobiletin has been shown to inhibit chikungunya (CHIKV) infection at viral entry as well as during translation/replication state. CHIKV exhibits an extraordinarily high morbidity rate of up to 97%, making the virus among the most challenging viruses to treat. Nobiletin has also been shown to increase the survival of virally infected cells 4-fold (increasing cell survival from <15% to −55%), while increasing cell survival 6-fold (to nearly 90% cell survival) when a higher concentration of nobiletin is used demonstrating decreased viral toxicity to the host cells as well as suppressing viral production. Nobiletin has also been shown to have direct anti-viral activity against enterovirus 71, as well as significant activity against hepatitis C virus. Therefore, administration of compositions comprising nobiletin may be both an effective prophylactic in the prevention of COVID-19 infection as well as an effective treatment when administered to a subject that has been infected with the SARS-CoV-2 virus.

Inflammation plays a significant role in determining the severity of COVID-19 symptoms and morbidity. In particular, COVID-19 morbidity is associated with the development of a “cytokine storm” in some patients. A cytokine storm is a hyperactive immune response characterized by the overproduction of cytokines, such as TNF-α, IL1-β, and IL-6. Nobiletin is a potent anti-inflammatory compound which may mitigate or prevent the inflammation and “cytokine storm” that is prevalent in serious COVID-19 infections. In particular, nobiletin has demonstrated significant anti-inflammatory effects by significantly inhibiting each one of the TNF-α, IL1-β, and IL-6 cytokines associated with lethal cytokine storms as well as other potent inflammatory biomarkers, such as NF-kB, IL1-α, MCP-1, toll-like receptor (TLR) 2+TLR4, along with inhibiting COX-2 (and its resulting pro-inflammatory metabolite, PGE₂). Additionally, nobiletin significantly reduces leukocyte infiltration into inflamed tissue, which is critical to the inflammatory modulation process. With tissue damage being one of the key issue related to severe COVID-19 infection, nobiletin can help protect healthy tissue by also inhibiting various collagenases (metalloproteinases), such as MMP-1, MMP-2, MMP-3, and MMP-9, while also upregulating TIMP-1, the body's natural inhibitor of MMP.

Obesity has been shown to be one of the factors that significantly increases the risk of serious COVID-19 symptoms. Nobiletin has several anti-obesity effects, including weight loss, fat loss, and the normalization of many of the metabolic defects arising from obesity. Although these are more chronic effects of nobiletin, they present relevance to both the prevention and treatment of COVID-19 symptoms.

Another one of the concerns of severe COVID-19 is thrombotic events. Nobiletin can exert significant anti-platelet activity, including the suppression of platelet aggregation. Therefore, administration of nobiletin to subjects suffering from COVID-19 may also mitigate thrombotic events in the subject.

Among the most important areas where nobiletin can be an effective treatment for COVID-19 is helping to maintain and/or restore healthy lung function, since the primary cause of death in severe COVID-19 is respiratory disease. Although this scenario is also typical of annual influenza, which can also lead to severe pneumonia and death, this process is far more robust and more lethal with COVID-19 infection. The typical process is that the influenza or SARS-CoV-2 infection can lead to pneumonia, which can lead to severe acute respiratory syndrome (SARS) and eventually to acute respiratory distress syndrome (ARDS) which is characterized by severe and widespread acute lung injury (ALI) to the point of interfering with the lungs' ability to take up oxygen. Administration of nobiletin, in a dose-dependent fashion, dramatically reduces induced lung damage, as seen in an acute lung injury research model, and does so in a highly effective manner, so much so that nobiletin nearly eliminated lung damage.

In each of the mechanisms described above by which nobiletin can significantly reduce COVID-19 infection and severity of related pathologies, its ability to perform these beneficial effects in vivo is greatly dependent on nobiletin's ability to penetrate the tissues of a subject, which is a unique feature of nobiletin. The enhanced ability of nobiletin to penetrate tissues is one of the reasons for consistent correlation between the demonstrated in vitro activity of nobiletin and its in vivo effects.

According to one aspect of the present disclosure, a composition for the treatment or prevention of COVID-19 infection in a subject is provided. The composition may include a nutritionally or pharmaceutically effective amount of nobiletin or a salt, solvate, or prodrug thereof. The composition may also be used for the treatment of a subject having and/or diagnosed with a respiratory infection or pneumonia caused by SARS-CoV-2. In at least some instances, the composition may further include one or more additional polymethoxylated flavones (PMFs). The one or more additional PMFs may be selected from the group consisting of tangeretin, sinensetin, and any combination thereof.

The presently disclosed compositions may have from about 2.5% to about 25% by weight nobiletin as compared to the total weight of the composition, or from about 1% to about 10% by weight nobiletin as compared to the total weight of the composition, or from about 0.7% to about 7.5% by weight nobiletin as compared to the total weight of the composition, or from about 0.7% to about 100% by weight nobiletin as compared to the total weight of the composition, or from about 0.25% to about 60% by weight nobiletin as compared to the total weight of the composition, or from about 0.2% to about 40% by weight nobiletin as compared to the total weight of the composition.

The composition may also include one or more omega-3 fatty acids. In some instances, the composition may include alpha-linolenic acid. In other instances, the composition may include at least one very long chain omega-3 fatty acid selected from the group consisting of eicosapentaenoic acid (EPA), docosapentaenoic acid (DPA), docosahexaenoic acid (DHA), and any combination thereof. In some cases, the composition may include alpha-linolenic acid (ALA) and at least one omega-3 fatty acid selected from the group consisting of EPA, DPA, and DHA. According to one aspect, the composition may also include at least one compound selected from the group consisting of natural triglycerides, restructured triglycerides, phospholipids, ethyl esters, free fatty acids, and any combination thereof.

The total weight of the one or more omega-3 fatty acids in the composition may be from about 12.5% to about 95% by weight of the total weight of the composition, or from about 5% to about 95% by weight of the total weight of the composition, or from about 3.5% to about 75% by weight of the total weight of the composition, or from about 25% to about 95% by weight of the total weight of the composition, or from about 10% to about 40% by weight of the total weight of the composition, or from about 7% to about 30% by weight of the total weight of the composition.

According to one aspect of the present disclosure, the composition may include EPA in an amount from about 12.5% to about 95% by weight of the total weight of the composition, or from about 5% to about 95% by weight of the total weight of the composition, or from about 3.5% to about 75% by weight of the total weight of the composition, or from about 25% to about 95% by weight of the total weight of the composition, or from about 10% to about 40% by weight of the total weight of the composition, or from about 7% to about 30% by weight of the total weight of the composition.

According to another aspect of the present disclosure, the composition may include DPA in an amount from about 12.5% to about 95% by weight of the total weight of the composition, or from about 5% to about 95% by weight of the total weight of the composition, or from about 3.5% to about 75% by weight of the total weight of the composition, or from about 25% to about 95% by weight of the total weight of the composition, or from about 10% to about 40% by weight of the total weight of the composition, or from about 7% to about 30% by weight of the total weight of the composition.

According to yet another aspect of the present disclosure, the composition may include DHA in an amount from about 12.5% to about 95% by weight of the total weight of the composition, or from about 5% to about 95% by weight of the total weight of the composition, or from about 3.5% to about 75% by weight of the total weight of the composition, or from about 25% to about 95% by weight of the total weight of the composition, or from about 10% to about 40% by weight of the total weight of the composition, or from about 7% to about 30% by weight of the total weight of the composition.

According to yet another aspect of the present disclosure, the composition may include ALA in an amount from about 12.5% to about 95% by weight of the total weight of the composition, or from about 5% to about 95% by weight of the total weight of the composition, or from about 3.5% to about 75% by weight of the total weight of the composition, or from about 25% to about 95% by weight of the total weight of the composition, or from about 10% to about 40% by weight of the total weight of the composition, or from about 7% to about 30% by weight of the total weight of the composition.

According to at least one aspect of the present disclosure, the composition may further include one or more natural anti-inflammatory botanic extracts selected from the group consisting of a turmeric extract, curcumin, a green tea (Camillia sinensis) extract, catechins, epigallocatechin gallate (EGCG), (−)-epicatechin, a Boswellia serrata extract, a ginger (Zingiber officinale) extract, gingerols, shogaols, a Scutellaria baicalensis extract, baicalein, caffeic acid phenethyl ester (CAPE), a cinnamon (Cinnamomum zeylanicum, Cinnamon cassia) extract, a Magnolia officinalis extract, honokiol, magnolol, Andrographis paniculata, Mangifera indica, magniferin, an olive (Olea europea) extract, oleuropein, hydroxytyrosol, verbascoside, Devil's Claw (Harpagophytum procumbens), hops (Humulus lupulus) extracts, Black seed (Nigella sativa) oil, thymoquinone, white willow (Salix alba) bark extract, salicin, and any combination thereof. The composition may also include one or more natural anti-inflammatory polyphenols selected from the group consisting of quercetin, isoquercitrin, fisetin, EGCG, (−)-epicatechin, luteolin, polymethoxylated luteolin, trans-resveratrol, pterostilbene, hesperidin, hesperitin, naringin, amentoflavone, chlorogenic acid, ursolic acid. The total weight of the one or more natural anti-inflammatory polyphenols in the composition may be from about 0.5% to about 25% by weight of the total weight of the composition, or from about 0.2% to about 10% by weight of the total weight of the composition, or from about 0.12% to about 7.5% by weight of the total weight of the composition, or from about 0.1% to about 85% by weight of the total weight of the composition, or from about 0.04% to about 40% by weight of the total weight of the composition, or from about 0.025% to about 30% by weight of the total weight of the composition.

The composition may also include one or more natural anti-inflammatory phytocannabinoids selected from the group consisting of a hemp (Cannabis sativa) extract, cannabidiol (CBD), oleolylethanolamide (OEA), palmitoylethanolamide (PEA), beta-caryophyllene, and any combination thereof.

The composition may also include one or more natural anti-inflammatory carotenoids selected from the group consisting of astaxanthin, lycopene, lutein, beta-carotene, zeaxanthin, and any combination thereof. The one or more natural anti-inflammatory carotenoids may comprise about 0.1% to about 2.5% by weight of the total weight of the composition, or from about 0.04% to about 1% by weight of the total weight of the composition, or from about 0.025% to about 0.75% by weight of the total weight of the composition, or from about 0.2% to about 1.2% by weight of the total weight of the composition, or from about 0.08% to about 0.5% by weight of the total weight of the composition, or from about 0.05% to about 0.35% by weight of the total weight of the composition.

According to one aspect, the composition may further include one or more compounds with immune modulating activity selected from the group consisting of beta-glucan, beta-1,3/1,6-glucan from yeast and beta-1,3-glucan mushrooms, mushroom extracts, a shiitake extract, a reishi (Ganoderma lucidum) extract, a maitake extract, an Echinacea purpurea and/or E. angustifolia extract, an elderberry (Sambucus nigra) extract, Andrographis paniculata, andrographolide, various algal extracts, a spirulina (Arthrospira platensis) extract, a chlorella (Chlorella vulgaris) extract, an Ecklonia cava extract, an Undaria pinnatifida extract, fucoidan, an Astragalus membranaceus extract, colostrum, milk powder from hyperimmunized cows, lactoferrin including apo-lactoferrin and/or partially iron saturated lactoferrin and/or iron-saturated lactoferrin, modified citrus pectin, and any combination thereof. The presently disclosed compositions may also include one or more essential nutrients with immune functions selected from the group consisting of vitamin C, vitamin D, zinc in all forms and chelates, selenium in all forms and chelates, and any combination thereof. In some instances, the compositions may include glutathione and/or one or more glutathione enhancing compounds selected from the group consisting of N-acetylcysteine (NAC), gamma-glutamylcysteine (GGC), (−)-epicatechin, selenium (preferably from high-selenium yeast), and any combination thereof.

In at least some instances, the presently disclosed compositions may also include one or more natural anti-virals selected from the group consisting of an elderberry (Sambucus nigra) extract, a mangosteen (Garcinia magnostana) extract, a Sutherlandia frutescens extract, dihydroquercetins (taxifolin and/or astilbin), a Undaria pinnafatida extract including fucoidan, lactoferrin including apo-lactoferrin and/or partially iron saturated lactoferrin and/or iron-saturated lactoferrin, zinc, selenium (preferably from high-selenium yeast), and any combination thereof. The compositions may also include one or more compounds with zinc ionophore activity selected from the group consisting of EGCG, quercetin, hydroxychloroquine, chloroquine, and any combination thereof. The presently disclosed compositions may also include one or more respiratory enhancing compounds selected from the group consisting of NAC, Pelargonium sidoides extract, serratiopeptidase, Jujube zizyphi, Picrorhiza kurroa, neem (Melia azadirachta) extract, perilla (Perilla frutescens) seed extract, luteolin, and any combination thereof.

In at least some aspects, the presently disclosed compositions may further include one or more cardiovascular support compounds selected from the group consisting of CoQ10, various hawthorne species (e.g., Crataegus oxyacantha) extracts, dansheng (Salvia miltiorrhiza) extract; cocoa extracts, including (−)-epicatechin; natural nitrates and/or purified nitrates; epimedium species (e.g., Epimedium sagittatum) extracts, including those containing the isolated monomers icariin and/or icariside II, osthole, L-arginine, L-citrulline, L-glutathione, and any combination thereof.

According to at least one aspect of the present disclosure, the presently disclosed compositions may be formulated into an oral pharmaceutical formulation. For instance, the compositions may be formulated as a powder, a capsule, tablet, softgel, microcapsule, a liquid suspension, and/or a liquid emulsion. In some instances, the presently disclosed compositions may be formulated into a topical pharmaceutical formulation such as a cream or ointment.

The presently disclosed formulations may be formulated to deliver to a subject in need thereof from about 7 mg to about 1400 mg nobiletin, or from about 25 mg to about 250 mg nobiletin, or from about 0.1 mg/kg body weight to about 20 mg/kg body weight nobiletin. The formulations may also be formulated to deliver to a subject in need thereof from about 125 mg to about 2500 mg omega-3 fatty acids, or from about 250 mg to about 1000 mg omega-3 fatty acids, or from about 125 mg to about 2500 mg EPA, or from about 250 mg to about 1000 mg EPA, or from about 125 mg to about 2500 mg DPA, or from about 250 mg to about 1000 mg DPA, or from about 125 mg to about 2500 mg DHA, or from about 250 mg to about 1000 mg DHA, or from about 125 mg to about 2500 mg ALA, or from about 250 mg to about 1000 mg ALA.

According to at least one aspect, the presently disclosed formulations may be formulated to deliver to a subject in need thereof from about 1 mg to about 1000 mg of one or more natural anti-inflammatory polyphenols, or from about 5 mg to about 250 mg of one or more natural anti-inflammatory polyphenols. The presently disclosed formulations may also be formulated to deliver to a subject in need thereof from about 1 mg to about 25 mg of one or more natural anti-inflammatory carotenoids, or from about 2 mg to about 12 mg of one or more natural anti-inflammatory carotenoids. The presently disclosed formulations may be formulated to deliver to a subject in need thereof from about 45 mg to about 8000 mg of vitamin C, or from about 90 mg to about 1000 mg of vitamin C, and/or from about 5 mcg to about 250 mcg of vitamin D, and/or from about 20 mcg to about 50 mcg of vitamin D, and/or from about 5.5 mg to about 75 mg of zinc, and/or from about 10 mg to about 25 mg of zinc, and/or from about 27.5 mcg to about 400 mcg of selenium, and/or from about 55 mcg to about 200 mcg of selenium.

According to an aspect of the present disclosure, a method for the treatment or prevention of COVID-19 infection in a subject is provided. The method may include administering to the subject a therapeutically effective amount of any of the presently disclosed compositions. Methods for the treatment of a subject infected with severe acute respiratory syndrome coronavirus 2 (SARS-CoV-2) and methods for the treatment of a subject having and/or diagnosed with a respiratory infection or pneumonia caused by severe acute respiratory syndrome coronavirus 2 (SARS-CoV-2) are also provided. The methods may include oral administration of one of the presently disclosed compositions in the form of an oral suspension or liquid to the subject. In other instances, the methods may include buccal, sublabial, or sublingual administration. In still other cases, administration may include administering one or more pills, tablets, capsules, or microcapsules. In some instances, one or more of the presently disclosed compositions may be administered by parenteral administration.

The presently disclosed methods may include administering to the subject a daily dose of from about 0.1 mg/kg body weight to about 20 mg/kg body weight nobiletin, or a daily dose of from about 7 mg to about 1400 mg nobiletin, or a daily dose of from about 25 mg to about 250 mg nobiletin. The presently disclosed methods may also include administering to the subject a daily dose of from about 125 mg to about 2500 mg omega-3 fatty acids, or administration of a daily dose of from about 250 mg to about 1000 mg omega-3 fatty acids, or administration of a daily dose of from about 125 mg to about 2500 mg EPA, or administration of a daily dose of from about 250 mg to about 1000 mg EPA, or administration of a daily dose of from about 125 mg to about 2500 mg DPA, or administration of a daily dose of from about 250 mg to about 1000 mg DPA, or administration of a daily dose of from about 125 mg to about 2500 mg DHA, or administration of a daily dose of from about 250 mg to about 1000 mg DHA, or administration of a daily dose of from about 125 mg to about 2500 mg ALA, or administration of a daily dose of from about 250 mg to about 1000 mg ALA.

The presently disclosed methods may also include administering to the subject a daily dose of from about 1 mg to about 1000 mg natural anti-inflammatory polyphenols, or administration of a daily dose of from about 5 mg to about 250 mg natural anti-inflammatory polyphenols and/or administration of a daily dose of from about 1 mg to about 25 mg natural anti-inflammatory carotenoids, or administration of a daily dose of from about 2 mg to about 12 mg natural anti-inflammatory carotenoids.

Alternatively, methods for the treatment or prevention of COVID-19 infection in a subject in need thereof are provided that include administering to the subject a therapeutically effective amount of one of the presently disclosed formulations. The presently disclosed formulations may also be used to treat a subject infected with severe acute respiratory syndrome coronavirus 2 (SARS-CoV-2), and for the treatment of a subject having and/or diagnosed with a respiratory infection or pneumonia caused by severe acute respiratory syndrome coronavirus 2 (SARS-CoV-2). In some instances, the subject may be a mammal. In other instances, the subject treated is a human.

### STATEMENTS OF THE DISCLOSURE

Numerous examples are provided herein to enhance understanding of the present disclosure. A specific set of statements are provided as follows.
Statement 1: A composition for the treatment or prevention of a viral respiratory infection, coronavirus infection, or COVID-19 infection in a subject, the composition comprising a nutritionally or pharmaceutically effective amount of nobiletin or a salt, solvate, or prodrug thereof.
Statement 2: A composition for the treatment of a subject having and/or diagnosed with a respiratory infection or pneumonia caused by severe acute respiratory syndrome coronavirus 2 (SARS-CoV-2), the composition comprising a nutritionally or pharmaceutically effective amount of nobiletin or a salt, solvate, or prodrug thereof.
Statement 3: A composition for the treatment or prevention of a viral respiratory infection, coronavirus infection, or COVID-19 infection in a subject, the composition comprising a nutritionally or pharmaceutically effective amount of 5,6,7,8,3′,4′-hexamethoxyflavone or a salt, solvate, or prodrug thereof
Statement 4: A composition for the treatment of a subject having and/or diagnosed with a respiratory infection or pneumonia caused by severe acute respiratory syndrome coronavirus 2 (SARS-CoV-2), the composition comprising a nutritionally or pharmaceutically effective amount of 5,6,7,8,3′,4′-hexamethoxyflavone or a salt, solvate, or prodrug thereof.
Statement 5: A composition for the treatment or prevention of a viral respiratory infection, coronavirus infection, or COVID-19 infection in a subject, the composition comprising a nutritionally or pharmaceutically effective amount of a compound according to structural formula I:

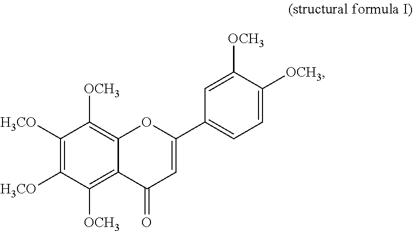 [SMILES: COc1ccc(-c2cc(=O)c3c(OC)c(OC)c(OC)c(OC)c3o2)cc1OC]

or a salt, solvate, or prodrug thereof
Statement 6: A composition for the treatment of a subject having and/or diagnosed with a respiratory infection or pneumonia caused by severe acute respiratory syndrome coronavirus 2 (SARS-CoV-2), the composition comprising a nutritionally or pharmaceutically effective amount of a compound according to structural formula I:

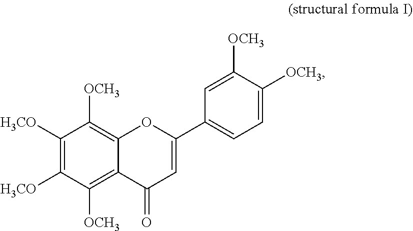 [SMILES: COc1ccc(-c2cc(=O)c3c(OC)c(OC)c(OC)c(OC)c3o2)cc1OC]

or a salt, solvate, or prodrug thereof
Statement 7: The composition according to any one of the preceding Statements 1-6, further comprising one or more additional polymethoxylated flavones (PMFs).
Statement 8: The composition according to Statement 7, wherein the one or more additional polymethoxylated flavones (PMFs) is selected from the group consisting of tangeretin, sinensetin, and any combination thereof
Statement 9: The composition according to any one of the preceding Statements 1-8, further comprising one or more omega-3 fatty acids.
Statement 10: The composition according to Statement 9, wherein the one or more omega-3 fatty acids comprises alpha-linolenic acid.
Statement 11: The composition according to Statement 9 or Statement 10, wherein the one or more omega-3 fatty acids comprises at least one very long chain omega-3 fatty acid selected from the group consisting of eicosapentaenoic acid (EPA), docosapentaenoic acid (DPA), docosahexaenoic acid (DHA), and any combination thereof.
Statement 12: The composition according to Statement 9, wherein the one or more omega-3 fatty acids comprises alpha-linolenic acid and at least one selected from the group consisting of eicosapentaenoic acid (EPA), docosapentaenoic acid (DPA), and docosahexaenoic acid (DHA).
Statement 13: The composition according to any one of the preceding Statements 1-12, further comprising at least one compound selected from the group consisting of natural triglycerides, restructured triglycerides, phospholipids, ethyl esters, free fatty acids, and any combination thereof
Statement 14: The composition according to any one of the preceding Statements 9-13, wherein the total weight of the one or more omega-3 fatty acids comprises from about 12.5% to about 95% by weight of the total weight of the composition.
Statement 15: The composition according to any one of the preceding Statements 9-13, wherein the total weight of the one or more omega-3 fatty acids comprises from about 5% to about 95% by weight of the total weight of the composition.
Statement 16: The composition according to any one of the preceding Statements 9-13, wherein the total weight of the one or more omega-3 fatty acids comprises from about 3.5% to about 75% by weight of the total weight of the composition.
Statement 17: The composition according to any one of the preceding Statements 9-13, wherein the total weight of the one or more omega-3 fatty acids comprises from about 25% to about 95% by weight of the total weight of the composition.
Statement 18: The composition according to any one of the preceding Statements 9-13, wherein the total weight of the one or more omega-3 fatty acids comprises from about 10% to about 40% by weight of the total weight of the composition.
Statement 19: The composition according to any one of the preceding Statements 9-13, wherein the total weight of the one or more omega-3 fatty acids comprises from about 7% to about 30% by weight of the total weight of the composition.
Statement 20: The composition according to any one of the preceding Statements 1-8, further comprising eicosapentaenoic acid (EPA).
Statement 21: The composition according to Statement 20, wherein the EPA comprises from about 12.5% to about 95% by weight of the total weight of the composition.
Statement 22: The composition according to Statement 20, wherein the EPA comprises from about 5% to about 95% by weight of the total weight of the composition.
Statement 23: The composition according to Statement 20, wherein the EPA comprises from about 3.5% to about 75% by weight of the total weight of the composition.
Statement 24: The composition according to Statement 20, wherein the EPA comprises from about 25% to about 95% by weight of the total weight of the composition.
Statement 25: The composition according to Statement 20, wherein the EPA comprises from about 10% to about 40% by weight of the total weight of the composition.
Statement 26: The composition according to Statement 20, wherein the EPA comprises from about 7% to about 30% by weight of the total weight of the composition.
Statement 27: The composition according to any one of the preceding Statements 1-8, further comprising docosapentaenoic acid (DPA).
Statement 28: The composition according to Statement 27, wherein the DPA comprises from about 12.5% to about 95% by weight of the total weight of the composition.
Statement 29: The composition according to Statement 27, wherein the DPA comprises from about 5% to about 95% by weight of the total weight of the composition.
Statement 30: The composition according to Statement 27, wherein the DPA comprises from about 3.5% to about 75% by weight of the total weight of the composition.
Statement 31: The composition according to Statement 27, wherein the DPA comprises from about 25% to about 95% by weight of the total weight of the composition.
Statement 32: The composition according to Statement 27, wherein the DPA comprises from about 10% to about 40% by weight of the total weight of the composition.
Statement 33: The composition according to Statement 27, wherein the DPA comprises from about 7% to about 30% by weight of the total weight of the composition.
Statement 34: The composition according to any one of the preceding Statements 1-8, further comprising docosahexaenoic acid (DHA).
Statement 35: The composition according to Statement 34, wherein the DHA comprises from about 12.5% to about 95% by weight of the total weight of the composition.
Statement 36: The composition according to Statement 34, wherein the DHA comprises from about 5% to about 95% by weight of the total weight of the composition.
Statement 37: The composition according to Statement 34, wherein the DHA comprises from about 3.5% to about 75% by weight of the total weight of the composition.
Statement 38: The composition according to Statement 34, wherein the DHA comprises from about 25% to about 95% by weight of the total weight of the composition.
Statement 39: The composition according to Statement 34, wherein the DHA comprises from about 10% to about 40% by weight of the total weight of the composition.
Statement 40: The composition according to Statement 34, wherein the DHA comprises from about 7% to about 30% by weight of the total weight of the composition.
Statement 41: The composition according to any one of the preceding Statements 1-40, further comprising one or more natural anti-inflammatory botanic extracts selected from the group consisting of a turmeric extract, curcumin; a green tea (Camillia sinensis) extract, catechins, epigallocatechin gallate (EGCG), (−)-epicatechin, a Boswellia serrata extract, a ginger (Zingiber officinale) extract, gingerols, shogaols, a Scutellaria baicalensis extract, baicalein, caffeic acid phenethyl ester (CAPE), a cinnamon (Cinnamomum zeylanicum, Cinnamon cassia) extract, a Magnolia officinalis extract, honokiol, magnolol, Andrographis paniculata, Mangifera indica, magniferin, an olive (Olea europea) extract, oleuropein, hydroxytyrosol, verbascoside, Devil's Claw (Harpagophytum procumbens), a hops (Humulus lupulus) extracts, black seed (Nigella sativa) oil, thymoquinone, white willow (Salix alba) bark extract, salicin, and any combination thereof.
Statement 42: The composition according to any one of the preceding Statements 1-41, further comprising one or more natural anti-inflammatory polyphenols selected from the group consisting of quercetin, isoquercitrin, fisetin, epigallocatechin gallate (EGCG), (−)-epicatechin, luteolin, polymethoxylated luteolin, trans-resveratrol, pterostilbene, hesperidin, hesperitin, naringin, amentoflavone, chlorogenic acid, ursolic acid.
Statement 43: The composition according to Statement 42, wherein the total weight of the one or more natural anti-inflammatory polyphenols comprises from about 0.5% to about 25% by weight of the total weight of the composition.
Statement 44: The composition according to Statement 42, wherein the total weight of the one or more natural anti-inflammatory polyphenols comprises from about 0.2% to about 10% by weight of the total weight of the composition.
Statement 45: The composition according to Statement 42, wherein the total weight of the one or more natural anti-inflammatory polyphenols comprises from about 0.12% to about 7.5% by weight of the total weight of the composition.
Statement 46: The composition according to Statement 42, wherein the total weight of the one or more natural anti-inflammatory polyphenols comprises from about 0.1% to about 85% by weight of the total weight of the composition.
Statement 47: The composition according to Statement 42, wherein the total weight of the one or more natural anti-inflammatory polyphenols comprises from about 0.04% to about 40% by weight of the total weight of the composition.
Statement 48: The composition according to Statement 42, wherein the total weight of the one or more natural anti-inflammatory polyphenols comprises from about 0.025% to about 30% by weight of the total weight of the composition.
Statement 49: The composition according to any one of the preceding Statements 1-48, further comprising one or more natural anti-inflammatory phytocannabinoids selected from the group consisting of a hemp (Cannabis sativa) extract, cannabidiol (CBD), oleolylethanolamide (OEA), palmitoylethanolamide (PEA), beta-caryophyllene, and any combination thereof.
Statement 50: The composition according to any one of the preceding Statements 1-49, further comprising one or more natural anti-inflammatory carotenoids selected from the group consisting of astaxanthin, lycopene, lutein, beta-carotene, zeaxanthin, and any combination thereof.
Statement 51: The composition according to Statement 50, wherein each of the one or more natural anti-inflammatory carotenoids comprises from about 0.1% to about 2.5% by weight of the total weight of the composition.
Statement 52: The composition according to Statement 50, wherein each of the one or more natural anti-inflammatory carotenoids comprises from about 0.04% to about 1% by weight of the total weight of the composition.
Statement 53: The composition according to Statement 50, wherein each of the one or more natural anti-inflammatory carotenoids comprises from about 0.025% to about 0.75% by weight of the total weight of the composition.
Statement 54: The composition according to Statement 50, wherein each of the one or more natural anti-inflammatory carotenoids comprises from about 0.2% to about 1.2% by weight of the total weight of the composition.
Statement 55: The composition according to Statement 50, wherein each of the one or more natural anti-inflammatory carotenoids comprises from about 0.08% to about 0.5% by weight of the total weight of the composition.
Statement 56: The composition according to Statement 50, wherein each of the one or more natural anti-inflammatory carotenoids comprises from about 0.05% to about 0.35% by weight of the total weight of the composition.
Statement 57: The composition according to any one of the preceding Statements 1-56, further comprising one or more compounds with immune modulating activity selected from the group consisting of beta-glucan, beta-1,3/1,6-glucan from yeast and beta-1,3-glucan from mushrooms, mushroom extracts, a shiitake extract, a reishi (Ganoderma lucidum) extract, a maitake extract, an Echinacea purpurea and/or E. angustifolia extract, an elderberry (Sambucus nigra) extract, Andrographis paniculata, andrographolide, various algal extracts, a spirulina (Arthrospira platensis) extract, a chlorella (Chlorella vulgaris) extract, an Ecklonia cava extract, an Undaria pinnatifida extract, fucoidan, an Astragalus membranaceus extract, colostrum, milk powder from hyperimmunized cows, lactoferrin including apo-lactoferrin and/or partially iron saturated lactoferrin and/or iron-saturated lactoferrin, modified citrus pectin, and any combination thereof.
Statement 58: The composition according to any one of the preceding Statements 1-57, further comprising one or more essential nutrients with immune functions selected from the group consisting of vitamin C, vitamin D, zinc in all forms and chelates, selenium in all forms and chelates, and any combination thereof.
Statement 59: The composition according to any one of the preceding Statements 1-58, further comprising glutathione and/or one or more glutathione enhancing compounds selected from the group consisting of N-acetylcysteine (NAC), gamma-glutamylcysteine (GGC), (−)-epicatechin, selenium (preferably from high-selenium yeast), and any combination thereof.
Statement 60: The composition according to any one of the preceding Statements 1-59, further comprising one or more natural anti-virals selected from the group consisting of an elderberry (Sambucus nigra) extract, a mangosteen (Garcinia magnostana) extract, a Sutherlandia frutescens extract, dihydroquercetins (taxifolin and/or astilbin), Undaria pinnafatida extract including fucoidan, lactoferrin including apo-lactoferrin and/or partially iron saturated lactoferrin and/or iron-saturated lactoferrin, zinc, selenium (preferably from high-selenium yeast), and any combination thereof.
Statement 61: The composition according to any one of the preceding Statements 1-60, further comprising one or more compounds with zinc ionophore activity selected from the group consisting of EGCG, quercetin, hydroxychloroquine, chloroquine, and any combination thereof
Statement 62: The composition according to any one of the preceding Statements 1-61, further comprising one or more respiratory enhancing compounds selected from the group consisting of N-acetylcysteine (NAC), Pelargonium sidoides extract, serratiopeptidase, Jujube zizyphi, Picrorhiza kurroa, neem (Melia azadirachta) extract, perilla (Perilla frutescens) seed extract, luteolin, and any combination thereof.
Statement 63: The composition according to any one of the preceding Statements 1-62, further comprising one or more cardiovascular support compounds selected from the group consisting of CoQ10, various hawthorne species (e.g., Crataegus oxyacantha) extracts, dansheng (Salvia miltiorrhiza) extract; cocoa extracts, including (−)-epicatechin; natural nitrates and/or purified nitrates; epimedium species (e.g., Epimedium sagittatum) extracts, including those containing the isolated monomers icariin and/or icariside II, osthole, L-arginine, L-citrulline, L-glutathione, and any combination thereof
Statement 64: The composition according to any one of the preceding Statements 1-63, comprising from about 2.5% to about 25% by weight nobiletin as compared to the total weight of the composition.
Statement 65: The composition according to any one of the preceding Statements 1-63, comprising from about 1% to about 10% by weight nobiletin as compared to the total weight of the composition.
Statement 66: The composition according to any one of the preceding Statements 1-63, comprising from about 0.7% to about 7.5% by weight nobiletin as compared to the total weight of the composition.
Statement 67: The composition according to any one of the preceding Statements 1-63, comprising from about 0.7% to about 100% by weight nobiletin as compared to the total weight of the composition.
Statement 68: The composition according to any one of the preceding Statements 1-63, comprising from about 0.25% to about 60% by weight nobiletin as compared to the total weight of the composition.
Statement 69: The composition according to any one of the preceding Statements 1-63, comprising from about 0.2% to about 40% by weight nobiletin as compared to the total weight of the composition.
Statement 70: An oral pharmaceutical formulation comprising any one of the compositions according to any one of the preceding Statements 1-69.
Statement 71: The oral pharmaceutical formulation according to Statement 70, formulated as a powder.
Statement 72: The oral pharmaceutical formulation according to Statement 70, formulated as a capsule, tablet, softgel, or microcapsule.
Statement 73: The oral pharmaceutical formulation according to Statement 70, formulated as a liquid suspension.
Statement 74: The oral pharmaceutical formulation according to Statement 70, formulated as a liquid emulsion.
Statement 75: A topical pharmaceutical formulation comprising any one of the compositions according to any one of the preceding Statements 1-69.
Statement 76: The pharmaceutical formulation according to any one of the preceding Statements 70-75, wherein the formulation is formulated to deliver to a subject in need thereof from about 7 mg to about 1400 mg nobiletin.
Statement 77: The pharmaceutical formulation according to any one of the preceding Statements 70-75, wherein the formulation is formulated to deliver to a subject in need thereof from about 25 mg to about 250 mg nobiletin.
Statement 78: The pharmaceutical formulation according to any one of the preceding Statements 70-75, wherein the formulation is formulated to deliver to a subject in need thereof from about 0.1 mg/kg body weight to about 20 mg/kg body weight nobiletin.
Statement 79: The pharmaceutical formulation according to any one of the preceding Statements 70-78, wherein the formulation is formulated to deliver to a subject in need thereof from about 125 mg to about 2500 mg omega-3 fatty acids.
Statement 80: The pharmaceutical formulation according to any one of the preceding Statements 70-78, wherein the formulation is formulated to deliver to a subject in need thereof from about 250 mg to about 1000 mg omega-3 fatty acids.
Statement 81: The pharmaceutical formulation according to any one of the preceding Statements 70-78, wherein the formulation is formulated to deliver to a subject in need thereof from about 125 mg to about 2500 mg EPA.
Statement 82: The pharmaceutical formulation according to any one of the preceding Statements 70-78, wherein the formulation is formulated to deliver to a subject in need thereof from about 250 mg to about 1000 mg EPA.
Statement 83: The pharmaceutical formulation according to any one of the preceding Statements 70-78, wherein the formulation is formulated to deliver to a subject in need thereof from about 125 mg to about 2500 mg DPA.
Statement 84: The pharmaceutical formulation according to any one of the preceding Statements 70-78, wherein the formulation is formulated to deliver to a subject in need thereof from about 250 mg to about 1000 mg DPA.
Statement 85: The pharmaceutical formulation according to any one of the preceding Statements 70-78, wherein the formulation is formulated to deliver to a subject in need thereof from about 125 mg to about 2500 mg DHA.
Statement 86: The pharmaceutical formulation according to any one of the preceding Statements 70-78, wherein the formulation is formulated to deliver to a subject in need thereof from about 250 mg to about 1000 mg DHA.
Statement 87: The pharmaceutical formulation according to any one of the preceding Statements 70-86, wherein the formulation is formulated to deliver to a subject in need thereof from about 1 mg to about 1000 mg of one or more natural anti-inflammatory polyphenols.
Statement 88: The pharmaceutical formulation according to any one of the preceding Statements 70-86, wherein the formulation is formulated to deliver to a subject in need thereof from about 5 mg to about 250 mg of one or more natural anti-inflammatory polyphenols.
Statement 89: The pharmaceutical formulation according to any one of the preceding Statements 70-88, wherein the formulation is formulated to deliver to a subject in need thereof from about 1 mg to about 25 mg of one or more natural anti-inflammatory carotenoids.
Statement 90: The pharmaceutical formulation according to any one of the preceding Statements 70-88, wherein the formulation is formulated to deliver to a subject in need thereof from about 2 mg to about 12 mg of one or more natural anti-inflammatory carotenoids.
Statement 91: The pharmaceutical formulation according to any one of the preceding Statements 70-90, wherein the formulation is formulated to deliver to a subject in need thereof from about 45 mg to about 8000 mg of vitamin C.
Statement 92: The pharmaceutical formulation according to any one of the preceding Statements 70-90, wherein the formulation is formulated to deliver to a subject in need thereof from about 90 mg to about 1000 mg of vitamin C.
Statement 93: The pharmaceutical formulation according to any one of the preceding Statements 70-91, wherein the formulation is formulated to deliver to a subject in need thereof from about 5 mcg to about 250 mcg of vitamin D.
Statement 94: The pharmaceutical formulation according to any one of the preceding Statements 70-91, wherein the formulation is formulated to deliver to a subject in need thereof from about 20 mcg to about 50 mcg of vitamin D.
Statement 95: The pharmaceutical formulation according to any one of the preceding Statements 70-94, wherein the formulation is formulated to deliver to a subject in need thereof from about 5.5 mg to about 75 mg of zinc.
Statement 96: The pharmaceutical formulation according to any one of the preceding Statements 70-94, wherein the formulation is formulated to deliver to a subject in need thereof from about 10 mg to about 25 mg of zinc.
Statement 97: The pharmaceutical formulation according to any one of the preceding Statements 70-96, wherein the formulation is formulated to deliver to a subject in need thereof from about 27.5 mcg to about 400 mcg of selenium.
Statement 98: The pharmaceutical formulation according to any one of the preceding Statements 70-96, wherein the formulation is formulated to deliver to a subject in need thereof from about 55 mcg to about 200 mcg of selenium.
Statement 99: A method for the treatment or prevention of COVID-19 infection in a subject in need thereof, the method comprising administering to the subject a therapeutically effective amount of the composition according to any one of the preceding Statements 1-69.
Statement 100: A method for the treatment of a subject infected with severe acute respiratory syndrome coronavirus 2 (SARS-CoV-2), the method comprising administering to the subject a therapeutically effective amount of the composition according to any one of the preceding Statements 1-69.
Statement 101: A method for the treatment of a subject having and/or diagnosed with a respiratory infection or pneumonia caused by severe acute respiratory syndrome coronavirus 2 (SARS-CoV-2), the method comprising administering to the subject a therapeutically effective amount of the composition according to any one of the preceding Statements 1-69.
Statement 102: The method according to any one of the preceding Statements 99-101, wherein the administering to the subject comprises oral administration.
Statement 103: The method according to Statement 102, wherein the oral administration comprises administering an oral suspension.
Statement 104: The method according to Statement 102, wherein the oral administration comprises administering a liquid to the subject.
Statement 105: The method according to Statement 102, wherein the oral administration comprises buccal, sublabial, or sublingual administration.
Statement 106: The method according to Statement 102, wherein the oral administration comprises administration of one or more pills, tablets, capsules, or microcapsules.
Statement 107: The method according to any one of the preceding Statements 99-101, wherein the administering to the subject comprises parenteral administration.
Statement 108: The method according to any one of the preceding Statements 99-107, wherein the administering to the subject comprises administration of a daily dose of from about 0.1 mg/kg body weight to about 20 mg/kg body weight nobiletin.
Statement 109: The method according to any one of the preceding Statements 99-107, wherein the administering to the subject comprises administration of a daily dose of from about 7 mg to about 1400 mg nobiletin.
Statement 110: The method according to any one of the preceding Statements 99-107, wherein the administering to the subject comprises administration of a daily dose of from about 25 mg to about 250 mg nobiletin.
Statement 111: The method according to any one of the preceding Statements 99-110, wherein the administering to the subject comprises administration of a daily dose of from about 125 mg to about 2500 mg omega-3 fatty acids.
Statement 112: The method according to any one of the preceding Statements 99-110, wherein the administering to the subject comprises administration of a daily dose of from about 250 mg to about 1000 mg omega-3 fatty acids.
Statement 113: The method according to any one of the preceding Statements 99-110, wherein the administering to the subject comprises administration of a daily dose of from about 125 mg to about 2500 mg EPA.
Statement 114: The method according to any one of the preceding Statements 99-110, wherein the administering to the subject comprises administration of a daily dose of from about 250 mg to about 1000 mg EPA.
Statement 115: The method according to any one of the preceding Statements 99-110, wherein the administering to the subject comprises administration of a daily dose of from about 125 mg to about 2500 mg DPA.
Statement 116: The method according to any one of the preceding Statements 99-110, wherein the administering to the subject comprises administration of a daily dose of from about 250 mg to about 1000 mg DPA.
Statement 117: The method according to any one of the preceding Statements 99-110, wherein the administering to the subject comprises administration of a daily dose of from about 125 mg to about 2500 mg DHA.
Statement 118: The method according to any one of the preceding Statements 99-110, wherein the administering to the subject comprises administration of a daily dose of from about 250 mg to about 1000 mg DHA.
Statement 119: The method according to any one of the preceding Statements 99-118, wherein the administering to the subject comprises administration of a daily dose of from about 1 mg to about 1000 mg natural anti-inflammatory polyphenols.
Statement 120: The method according to any one of the preceding Statements 99-118, wherein the administering to the subject comprises administration of a daily dose of from about 5 mg to about 250 mg natural anti-inflammatory polyphenols.
Statement 121: The method according to any one of the preceding Statements 99-120, wherein the administering to the subject comprises administration of a daily dose of from about 1 mg to about 25 mg natural anti-inflammatory carotenoids.
Statement 122: The method according to any one of the preceding Statements 99-120, wherein the administering to the subject comprises administration of a daily dose of from about 2 mg to about 12 mg natural anti-inflammatory carotenoids.
Statement 123: The method according to any one of the preceding Statements 99-122, wherein the administering to the subject comprises administration of a daily dose of from about 45 mg to about 8000 mg vitamin C.
Statement 124: The method according to any one of the preceding Statements 99-122, wherein the administering to the subject comprises administration of a daily dose of from about 90 mg to about 1000 mg vitamin C.
Statement 125: The method according to any one of the preceding Statements 99-124, wherein the administering to the subject comprises administration of a daily dose of from about 5 mcg to about 250 mcg vitamin D.
Statement 126: The method according to any one of the preceding Statements 99-124, wherein the administering to the subject comprises administration of a daily dose of from about 20 mcg to about 50 mcg vitamin D.
Statement 127: The method according to any one of the preceding Statements 99-126, wherein the administering to the subject comprises administration of a daily dose of from about 5.5 mg to about 75 mg zinc.
Statement 128: The method according to any one of the preceding Statements 99-126, wherein the administering to the subject comprises administration of a daily dose of from about 10 mg to about 25 mg zinc.
Statement 129: The method according to any one of the preceding Statements 99-128, wherein the administering to the subject comprises administration of a daily dose of from about 27.5 mcg to about 400 mcg selenium.
Statement 130: The method according to any one of the preceding Statements 99-128, wherein the administering to the subject comprises administration of a daily dose of from about 55 mcg to about 200 mcg selenium.
Statement 131: The composition according to any one of the preceding Statements 1-8 and 41-69, further comprising alpha-linolenic acid (ALA).
Statement 132: The composition according to Statement 131, wherein the ALA comprises from about 12.5% to about 95% by weight of the total weight of the composition.
Statement 133: The composition according to Statement 131, wherein the ALA comprises from about 5% to about 95% by weight of the total weight of the composition.
Statement 134: The composition according to Statement 131, wherein the ALA comprises from about 3.5% to about 75% by weight of the total weight of the composition.
Statement 135: The composition according to Statement 131, wherein the ALA comprises from about 25% to about 95% by weight of the total weight of the composition.
Statement 136: The composition according to Statement 131, wherein the ALA comprises from about 10% to about 40% by weight of the total weight of the composition.
Statement 137: The composition according to Statement 131, wherein the ALA comprises from about 7% to about 30% by weight of the total weight of the composition.
Statement 138: An oral pharmaceutical formulation comprising any one of the compositions according to any one of the preceding Statements 131-137.
Statement 139: The oral pharmaceutical formulation according to Statement 138, formulated as a powder.
Statement 140: The oral pharmaceutical formulation according to Statement 138, formulated as a capsule, tablet, softgel, or microcapsule.
Statement 141: The oral pharmaceutical formulation according to Statement 138, formulated as a liquid suspension.
Statement 142: The oral pharmaceutical formulation according to Statement 138, formulated as a liquid emulsion.
Statement 143: A topical pharmaceutical formulation comprising any one of the compositions according to any one of the preceding Statements 131-137.
Statement 144: A method for the treatment or prevention of COVID-19 infection in a subject in need thereof, the method comprising administering to the subject a therapeutically effective amount of the composition according to any one of the preceding Statements 131-137.
Statement 145: A method for the treatment of a subject infected with severe acute respiratory syndrome coronavirus 2 (SARS-CoV-2), the method comprising administering to the subject a therapeutically effective amount of the composition according to any one of the preceding Statements 131-137.
Statement 146: A method for the treatment of a subject having and/or diagnosed with a respiratory infection or pneumonia caused by severe acute respiratory syndrome coronavirus 2 (SARS-CoV-2), the method comprising administering to the subject a therapeutically effective amount of the composition according to any one of the preceding Statements 131-137.
Statement 147: The method according to any one of the preceding Statements 144-146, wherein the administering to the subject comprises administration of a daily dose of from about 125 mg to about 2500 mg ALA.
Statement 148: The method according to any one of the preceding Statements 144-146, wherein the administering to the subject comprises administration of a daily dose of from about 250 mg to about 1000 mg ALA.
Statement 149: A method for the treatment or prevention of COVID-19 infection in a subject in need thereof, the method comprising administering to the subject a therapeutically effective amount of the formulation according to any one of the preceding Statements 70-98 and 138-143.
Statement 150: A method for the treatment of a subject infected with severe acute respiratory syndrome coronavirus 2 (SARS-CoV-2), the method comprising administering to the subject a therapeutically effective amount of the formulation according to any one of the preceding Statements 70-98 and 138-143.
Statement 151: A method for the treatment of a subject having and/or diagnosed with a respiratory infection or pneumonia caused by severe acute respiratory syndrome coronavirus 2 (SARS-CoV-2), the method comprising administering to the subject a therapeutically effective amount of the formulation according to any one of the preceding Statements 70-98 and 138-143.
Statement 152: The method according to any one of the preceding Statements 99-133 and 144-151, wherein the subject is a mammal.
Statement 153: The method according to any one of the preceding Statements 99-133 and 144-151, wherein the subject is a human.
Statement 154: A composition for the treatment or prevention of a viral respiratory infection in a subject, the composition comprising a nutritionally or pharmaceutically effective amount of nobiletin or a salt, solvate, or prodrug thereof.
Statement 155: A composition for the treatment of a subject having and/or diagnosed with a pneumonia caused by a viral respiratory infection, the composition comprising a nutritionally or pharmaceutically effective amount of nobiletin or a salt, solvate, or prodrug thereof.
Statement 156: A composition for the treatment or prevention of a viral respiratory infection in a subject, the composition comprising a nutritionally or pharmaceutically effective amount of 5,6,7,8,3′,4′-hexamethoxyflavone or a salt, solvate, or prodrug thereof.
Statement 157: A composition for the treatment of a subject having and/or diagnosed with pneumonia caused by a viral respiratory infection, the composition comprising a nutritionally or pharmaceutically effective amount of 5,6,7,8,3′,4′-hexamethoxyflavone or a salt, solvate, or prodrug thereof
Statement 158: A composition for the treatment or prevention of a viral respiratory infection in a subject, the composition comprising a nutritionally or pharmaceutically effective amount of a compound according to structural formula I:

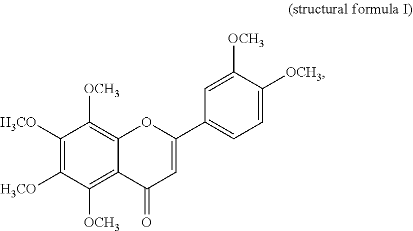 [SMILES: COc1ccc(-c2cc(=O)c3c(OC)c(OC)c(OC)c(OC)c3o2)cc1OC]

or a salt, solvate, or prodrug thereof
Statement 159: A composition for the treatment of a subject having and/or diagnosed with pneumonia caused by a viral respiratory infection, the composition comprising a nutritionally or pharmaceutically effective amount of a compound according to structural formula I:

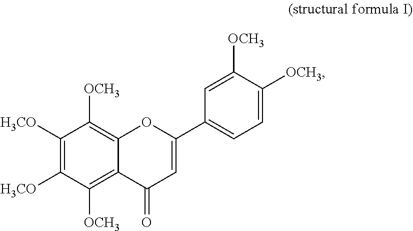 [SMILES: COc1ccc(-c2cc(=O)c3c(OC)c(OC)c(OC)c(OC)c3o2)cc1OC]

or a salt, solvate, or prodrug thereof.
Statement 160: The composition according to any one of the preceding Statements 154-159, further comprising one or more additional polymethoxylated flavones (PMFs).
Statement 161: The composition according to Statement 160, wherein the one or more additional polymethoxylated flavones (PMFs) is selected from the group consisting of tangeretin, sinensetin, and any combination thereof
Statement 162: The composition according to any one of the preceding Statements 154-161, further comprising one or more omega-3 fatty acids.
Statement 163: The composition according to Statement 162, wherein the one or more omega-3 fatty acids comprises alpha-linolenic acid.
Statement 164: The composition according to Statement 162 or Statement 163, wherein the one or more omega-3 fatty acids comprises at least one very long chain omega-3 fatty acid selected from the group consisting of eicosapentaenoic acid (EPA), docosapentaenoic acid (DPA), docosahexaenoic acid (DHA), and any combination thereof.
Statement 165: The composition according to Statement 162, wherein the one or more omega-3 fatty acids comprises alpha-linolenic acid and at least one selected from the group consisting of eicosapentaenoic acid (EPA), docosapentaenoic acid (DPA), and docosahexaenoic acid (DHA).
Statement 166: The composition according to any one of the preceding Statements 154-165, further comprising at least one compound selected from the group consisting of natural triglycerides, restructured triglycerides, phospholipids, ethyl esters, free fatty acids, and any combination thereof
Statement 167: The composition according to any one of the preceding Statements 154-166, comprising from about 2.5% to about 25% by weight nobiletin as compared to the total weight of the composition.
Statement 168: The composition according to any one of the preceding Statements 154-166, comprising from about 1% to about 10% by weight nobiletin as compared to the total weight of the composition.
Statement 169: The composition according to any one of the preceding Statements 154-166, comprising from about 0.7% to about 7.5% by weight nobiletin as compared to the total weight of the composition.
Statement 170: The composition according to any one of the preceding Statements 154-166, comprising from about 0.7% to about 100% by weight nobiletin as compared to the total weight of the composition.
Statement 171: The composition according to any one of the preceding Statements 154-166, comprising from about 0.25% to about 60% by weight nobiletin as compared to the total weight of the composition.
Statement 172: The composition according to any one of the preceding Statements 154-166, comprising from about 0.2% to about 40% by weight nobiletin as compared to the total weight of the composition.
Statement 173: The composition according to any one of the preceding Statements 154-172, wherein the viral respiratory infection is the common cold.
Statement 174: The composition according to any one of the preceding Statements 154-172, wherein the viral respiratory infection is influenza A.
Statement 175: The composition according to any one of the preceding Statements 154-172, wherein the viral respiratory infection is influenza B.
Statement 176: The composition according to any one of the preceding Statements 154-172, wherein the viral respiratory infection is caused by a coronavirus.
Statement 177: A method for the treatment or prevention of a viral respiratory infection in a subject in need thereof, the method comprising administering to the subject a therapeutically effective amount of the composition according to any one of the preceding Statements 154-176.
Statement 178: A method for the treatment of a subject having and/or diagnosed with a pneumonia caused by a viral respiratory infection, the method comprising administering to the subject a therapeutically effective amount of the composition according to any one of the preceding Statements 154-137.
Statement 179: The method according to Statement 177 or Statement 178, wherein the viral respiratory infection is the common cold.
Statement 180: The method according to Statement 177 or Statement 178, wherein the viral respiratory infection is influenza A.
Statement 181: The method according to Statement 177 or Statement 178, wherein the viral respiratory infection is influenza B.
Statement 182: The method according to Statement 177 or Statement 178, wherein the viral respiratory infection is caused by a coronavirus.
Statement 183: The method according to any one of the preceding Statements 177-182, wherein the subject is a mammal.
Statement 184: The method according to any one of the preceding Statements 177-182, wherein the subject is a human.
Statement 185: A composition for the treatment or prevention of a viral respiratory infection in a subject, the composition comprising a nutritionally or pharmaceutically effective amount of nobiletin or a salt, solvate, or prodrug thereof.
Statement 186: The composition according to Statement 185, wherein the subject has and/or is diagnosed with a pneumonia caused by a viral respiratory infection.
Statement 187: The composition according to Statement 185 or Statement 186, wherein the viral respiratory infection is selected from the group consisting of the common cold, influenza A, influenza B, a coronavirus, severe acute respiratory syndrome coronavirus 2 (SARS-CoV-2), and any combination thereof
Statement 188: The composition according to Statement 187, wherein the viral respiratory infection is severe acute respiratory syndrome coronavirus 2 (SARS-CoV-2).
Statement 189: The composition according to any one of the preceding Statements 185-188, further comprising one or more additional polymethoxylated flavones (PMFs).
Statement 190: The composition according to Statement 189, wherein the one or more additional polymethoxylated flavones (PMFs) is selected from the group consisting of tangeretin, sinensetin, and any combination thereof
Statement 191: The composition according to any one of the preceding Statements 185-189, further comprising one or more omega-3 fatty acids.
Statement 192: The composition according to Statement 191, wherein the one or more omega-3 fatty acids comprises alpha-linolenic acid.
Statement 193: The composition according to Statement 191, wherein the one or more omega-3 fatty acids comprises at least one very long chain omega-3 fatty acid selected from the group consisting of eicosapentaenoic acid (EPA), docosapentaenoic acid (DPA), docosahexaenoic acid (DHA), and any combination thereof.
Statement 194: The composition according to Statement 191, wherein the one or more omega-3 fatty acids comprises alpha-linolenic acid and at least one selected from the group consisting of eicosapentaenoic acid (EPA), docosapentaenoic acid (DPA), and docosahexaenoic acid (DHA).
Statement 195: The composition according to any one of the preceding Statements 185-194, further comprising at least one compound selected from the group consisting of natural triglycerides, restructured triglycerides, phospholipids, ethyl esters, free fatty acids, and any combination thereof
Statement 196: The composition according to any one of the preceding Statements 185-195, comprising from about 0.25% to about 60% by weight nobiletin as compared to the total weight of the composition.
Statement 197: A method for the treatment or prevention of a viral respiratory infection in a subject in need thereof, the method comprising administering to the subject a therapeutically effective amount of a composition, the composition comprising nobiletin or a salt, solvate, or prodrug thereof
Statement 198: The method according to Statement 197, wherein the subject has and/or is diagnosed with a pneumonia caused by a viral respiratory infection.
Statement 199: The method according to Statement 197 or Statement 198, wherein the viral respiratory infection is selected from the group consisting of the common cold, influenza A, influenza B, a coronavirus, severe acute respiratory syndrome coronavirus 2 (SARS-CoV-2), and any combination thereof.
Statement 200: The method according to Statement 197 or Statement 198, wherein the viral respiratory infection is severe acute respiratory syndrome coronavirus 2 (SARS-CoV-2).
Statement 201: The method according to any one of the preceding Statements 197-200, wherein the composition further comprises one or more additional polymethoxylated flavones (PMFs).
Statement 202: The method according to Statement 201, wherein the one or more additional polymethoxylated flavones (PMFs) is selected from the group consisting of tangeretin, sinensetin, and any combination thereof
Statement 203: The method according to any one of the preceding Statements 197-203, wherein the composition further comprises one or more omega-3 fatty acids.
Statement 204: The method according to Statement 203, wherein the one or more omega-3 fatty acids comprises alpha-linolenic acid.
Statement 205: The method according to Statement 203, wherein the one or more omega-3 fatty acids comprises at least one very long chain omega-3 fatty acid selected from the group consisting of eicosapentaenoic acid (EPA), docosapentaenoic acid (DPA), docosahexaenoic acid (DHA), and any combination thereof.
Statement 206: The method according to Statement 203, wherein the one or more omega-3 fatty acids comprises alpha-linolenic acid and at least one selected from the group consisting of eicosapentaenoic acid (EPA), docosapentaenoic acid (DPA), and docosahexaenoic acid (DHA).
Statement 207: The method according to any one of the preceding Statements 197-206, wherein the composition further comprises at least one compound selected from the group consisting of natural triglycerides, restructured triglycerides, phospholipids, ethyl esters, free fatty acids, and any combination thereof.
Statement 208: The method according to any one of the preceding Statements 197-207, wherein the composition comprises from about 0.25% to about 60% by weight nobiletin as compared to the total weight of the composition.
Statement 209: The method according to any one of the preceding Statements 197-208, wherein the subject is a mammal.
Statement 210: The method according to any one of the preceding Statements 197-208, wherein the subject is a human.

What is claimed is:

1.-26. (canceled)

27. A composition for the treatment or prevention of a viral respiratory infection in a subject, the composition comprising a nutritionally or pharmaceutically effective amount of nobiletin or a salt, solvate, or prodrug thereof.

28. The composition according to claim 27, wherein the subject has and/or is diagnosed with a pneumonia caused by a viral respiratory infection.

29. The composition according to claim 27 or claim 28, wherein the viral respiratory infection is selected from the group consisting of the common cold, influenza A, influenza B, a coronavirus, severe acute respiratory syndrome coronavirus 2 (SARS-CoV-2), and any combination thereof.

30. The composition according to claim 27, further comprising one or more additional polymethoxylated flavones (PMFs) selected from the group consisting of tangeretin, sinensetin, and any combination thereof.

31. The composition according to claim 27, further comprising one or more omega-3 fatty acids.

32. The composition according to claim 31, wherein the one or more omega-3 fatty acids comprises alpha-linolenic acid.

33. The composition according to claim 31, wherein the one or more omega-3 fatty acids comprises at least one very long chain omega-3 fatty acid selected from the group consisting of eicosapentaenoic acid (EPA), docosapentaenoic acid (DPA), docosahexaenoic acid (DHA), and any combination thereof.

34. The composition according to claim 31, wherein the one or more omega-3 fatty acids comprises alpha-linolenic acid and at least one selected from the group consisting of eicosapentaenoic acid (EPA), docosapentaenoic acid (DPA), and docosahexaenoic acid (DHA).

35. The composition according to claim 27, further comprising at least one compound selected from the group consisting of natural triglycerides, restructured triglycerides, phospholipids, ethyl esters, free fatty acids, and any combination thereof.

36. The composition according to claim 27, comprising from about 0.25% to about 60% by weight nobiletin as compared to the total weight of the composition.

37. A method for the treatment or prevention of a viral respiratory infection in a subject in need thereof, the method comprising administering to the subject a therapeutically effective amount of a composition, the composition comprising nobiletin or a salt, solvate, or prodrug thereof.

38. The method according to claim 37, wherein the subject has and/or is diagnosed with a pneumonia caused by a viral respiratory infection.

39. The method according to claim 37 or claim 38, wherein the viral respiratory infection is selected from the group consisting of the common cold, influenza A, influenza B, a coronavirus, severe acute respiratory syndrome coronavirus 2 (SARS-CoV-2), and any combination thereof.

40. The method according to claim 39, wherein the composition further comprises one or more additional polymethoxylated flavones (PMFs) selected from the group consisting of tangeretin, sinensetin, and any combination thereof.

41. The method according to claim 39, wherein the composition further comprises one or more omega-3 fatty acids.

42. The method according to claim 41, wherein the one or more omega-3 fatty acids comprises alpha-linolenic acid.

43. The method according to claim 41, wherein the one or more omega-3 fatty acids comprises at least one very long chain omega-3 fatty acid selected from the group consisting of eicosapentaenoic acid (EPA), docosapentaenoic acid (DPA), docosahexaenoic acid (DHA), and any combination thereof.

44. The method according to claim 41, wherein the one or more omega-3 fatty acids comprises alpha-linolenic acid and at least one selected from the group consisting of eicosapentaenoic acid (EPA), docosapentaenoic acid (DPA), and docosahexaenoic acid (DHA).

45. The method according to claim 41, wherein the composition further comprises at least one compound selected from the group consisting of natural triglycerides, restructured triglycerides, phospholipids, ethyl esters, free fatty acids, and any combination thereof.

46. The method according to claim 37, wherein the composition comprises from about 0.25% to about 60% by weight nobiletin as compared to the total weight of the composition.
